(19) United States
(12) Patent Application Publication
(10) Pub. No.: US 2011/0318779 A1
(43) Pub. Date: Dec. 29, 2011

(54) PROCESS FOR PRODUCING RECOMBINANT PROTEIN USING NOVEL FUSION PARTNER

(75) Inventors: Hang-Cheol Shin, Seoul (KR); Seung-Hwan Jang, Gyeonggi-do (KR); Eun-Hye Ko, Incheon (KR); Hyo-Jin Kim, Gyeonggi-do (KR); Yean-Hee Park, Seoul (KR); Myung-Hwan Kim, Seoul (KR); Ki-Hoon Yoon, Gyeonggi-do (KR); Hyang-Do Song, Seoul (KR); Hye-Ran Hyun, Seoul (KR)

(73) Assignee: Nutrex Technology Co., Ltd., Seocho-gu, Seoul (KR)

(21) Appl. No.: 12/735,242

(22) PCT Filed: Dec. 24, 2007
(86) PCT No.: PCT/KR2007/006792

§ 371 Date: Jun. 24, 2010

(51) Int. Cl. C12P 21/06; C07K 19/00; C12P 21/02; C12N 15/63; C12N 1/21

(52) U.S. Cl. 435/68.1; 435/69.4; 435/320.1; 435/252.33; 435/69.7; 530/350; 530/399; 530/397

(57) ABSTRACT

The present invention provides a method of producing polypeptide utilizing a fusion protein of A-B type in the following formula (I), by culturing transformed microorganism comprising DNA sequence encoding the desirable polypeptide; A-B (I). In the above formula (I), A is a fusion partner of 25 or more amino acid residues where aspartic and glutamic acid residues are incorporated to have a net negative charge of 30% or more, and B is the target protein to be produced. The target protein can be isolated from the fusion protein by employing enzymatic cleavage site etc. at the carboxyl-terminus of the fusion partner.

### TECHNICAL FIELD

This invention relates to a new method for the production of recombinant proteins using novel fusion partners.

### BACKGROUND ART

At the advent of new technologies of genetic recombination, biologically useful proteins have been produced utilizing prokaryotes (Escherichia coli), yeast (Saccharomyces cerevisiae), and cells of higher organisms. These recombinant proteins are widely used in the biotechnology industries for therapeutics and other biologicals for various purposes. In particular, Escherichia coli (E. coli) is the most preferred host cells for recombinant protein production due to the fast growth rate and the well-defined molecular biology.

The protein production systems using E. coli have an excellent economic efficiency in view of the cost, equipments and process operations, but they have one major problem in which a majority of foreign proteins are produced inside the cell as inactive Inclusion Body (IB) and require a refolding process to obtain an active folded structure. In order to obtain an active form from the IB, the IB should be solubilized in a high concentration of guanidine hydrochloride (GdnHCl) or urea, and then refolded into the native structure using methods such as dilution, etc. The refolding mechanism is not well understood and the refolding process of each protein depends on the inherent and unique characteristics of proteins themselves. This inherent problem is the cause of low yield, high production cost, and prolonging time [Lilie, H. et al. (1998) Curr. Opin. Biotechnol. 9, 497-501], and it is difficult or impossible to refold a majority of high molecular weight proteins, which is an obstacle to industrial applications of the proteins.

The formation of IB is characterized by a competition of intramolecular folding rate and intermolecular aggregation. In case where intramolecular folding rate is slower than the rate of intermolecular aggregation, aggregates in the form of IB are formed [Mitraki, A. & King, J. (1989) Bio/Technology 7, 690-697].

The present invention is to use specific peptide sequences of 30% or higher ratio of negative charges as fusion partners to prevent the formation of aggregates, hence to obtain correctly folded soluble form. The aggregation of folding intermediates is effectively inhibited by the intermolecular repulsion between the negative charges existing in the fusion partners, thereby dramatic improvement of producing native proteins can be achieved.

When the fusion partners of this invention are applied to the manufacturing process of insulin, formation of IB has been effectively eliminated and native insulin has been produced after a simple oxidation in buffer solution.

Human insulin is currently produced either in E. coli or in yeast using recombinant technology [Frank, B. H et al. (1981) In: Peptides: Synthesis-Structure-function (ed. Rich, D. H. Gross, E.) pp. 729-738, Proceedings of the Seventh American Peptide Symposium, Pierce Chemical Co., Rockford, Ill.; Thim, L. et al. (1986) Proc. Natl. Acad. Sci. USA 83, 6766-6770; Markussen, J. et al (1987) Protein Engineering 1, 205-213]

The production of human insulin in E. coli uses either proinsulin (PI) [Frank, B. H et al. (1981) In: Peptides: Synthesis-Structure-function (ed. Rich, D. H. Gross, E.) pp. 729-738, Proceedings of the Seventh American Peptide Symposium, Pierce Chemical Co., Rockford, Ill.] or miniproinsulin (mini-PI) [Chang, S.-G. et al. (1998) Biochem J. 329, 631-635] as a precursor. First, PI or mini-PI in the form of fusion protein is produced as IB, and then the IB are solubilized in denaturing agents such as urea or GdnHCl. The PI or mini-PI as a sulfonated form is isolated from the fusion protein by means of cyanogen bromide (CNBr) cleavage, sulfonation and purification steps. The sulfonated PI or mini-PI is refolded into the native form, and the insulin is produced by treatment of trypsin and carboxypeptidase B followed by several purification steps. Refolding yield of PI or mini-PI is greatly influenced by the concentration of the refolding proteins, showing lower yields at higher concentrations. The downstream process including solubilization, CNBr cleavage and sulfonation takes up most of expenditures leading to high cost of production. The CNBr cleavage in particular results in a 50-60% poor production yield and is recognized as a technology to overcome.

A method of reducing the process steps after fermentation is to use yeast cells, in which insulin is produced through a series of downstream process (enzymatic reaction, acid hydrolysis, isolation and purification) after extracellular secretion of a single chain insulin derivative. The production yield is low, but the yeast system makes purification step easier and does not require refolding steps, a difficult process experienced in the prokaryotes [Thim, L. et al. (1986) Proc. Natl, Acad. Sci, USA 83, 6766-6770].

Therefore, the combination of high expression level of E. coli with elimination of cumbersome refolding process envisaged in yeast system would be an excellent choice for the manufacturing process of insulin.

### DISCLOSURE OF INVENTION

This invention provides a method of producing polypeptide utilizing a fusion protein of A-B type in the following formula (I), by culturing transformed microorganism containing DNA sequence encoding the polypeptide;

A-B (I)

In the above formula (I), A is a fusion partner of 25 or more amino acid residues where aspartic and glutamic acid residues are incorporated to have a net negative charge of 30% or more, and B is the target protein to be produced.

As demonstrated in the Embodiments, it is preferable that A of A-B in (I) is a peptide, wherein part of the peptide comprises a sequence of 7 consecutive amino acid residues with 5 or more negative charges;

It is also preferable that A of A-B in (I) is a peptide comprising MKIEEGKL sequence at the amino-terminus;

It is also preferable that A of A-B in (I) is one of peptides of the following SEQ ID NO: 64 to SEQ ID NO: 74, but is not limited thereto.

SEQ ID NO: 64:
MGSSHHHHHHSSGLVPRGSDMAGDNDDLDLEEALEPDMEEDDDQ
SEQ ID NO: 65:
MAGDNDDLDLEEALEPDMEEDDDQ
SEQ ID NO: 66:
MKIEEGKLAGDNDDLDLEEALEPDMEEDDDQ
SEQ ID NO: 67:
MSEQHAQGAGDNDDLDLEEALEPDMEEDDDQ
SEQ ID NO: 68:
MKIEEGKLAGDNDDLDLEEALEPDME
SEQ ID NO: 69:
MSEQHAQGAGDNDDLDLEEALEPDME
SEQ ID NO: 70:
MKIEEGKLEALEPDMEEDDDQ
SEQ ID NO: 71:
MSEQHAQGEALEPDMEEDDDQ
SEQ ID NO: 72:
MKIEEGKLAGDNDDLDLEEAL
SEQ ID NO: 73:
MSEQHAQGAGDNDDLDLEEAL
SEQ ID NO: 74:
MGSSHHHHHHSSAGDNDDLDLEEALEPDMEEDDDQ

In addition, it is possible to isolate the target protein from the fusion protein by incorporating enzymatic cleavage site to the carboxyl-terminus of the fusion partner A.

Furthermore, this invention provides a method of producing Proinsulin (PI) or analogues according to the method of producing polypeptide of the present invention. The invention also provides a method of producing insulin further comprising procedures for enzymatic hydrolysis or chemical cleavage after the preparation of PI and its analogues.

The invention provides a pharmaceutical composition comprising the insulin produced by the method of the present invention and a pharmaceutically acceptable carrier.

The invention provides a method of producing granulocyte colony stimulating factor (GCSF) or its analogues according to the manufacturing method of this invention.

The invention provides a pharmaceutical composition comprising the granulocyte colony stimulating factor (GCSF) produced by the method of the present invention and a pharmaceutically acceptable carrier.

The invention also provides method of producing growth hormone (GH) or its analogues according to the manufacturing method of this invention.

The invention provides a pharmaceutical composition comprising the growth hormone (GH) produced by the method of the present invention and a pharmaceutically acceptable carrier.

The invention also provides method of producing bone morphogenetic protein 2 (BMP2) or its analogues according to the manufacturing method of this invention.

The invention provides a pharmaceutical composition comprising the bone morphogenetic protein 2 (BMP2) produced by the method of the present invention and a pharmaceutically acceptable carrier.

Also, this invention provides a fusion protein of A-B type in the following formula (I);

A-B (I)

In the above (I), A is a fusion partner of 25 or more amino acid residues where aspartic and glutamic acid residues are incorporated to have a net negative charge of 30% or more, and B is the target protein to be produced.

As demonstrated in the Embodiments, it is preferable that A of A-B in (I) is a peptide, wherein part of the peptide comprises a sequence of 7 consecutive amino acid residues with 5 or more negative charges;

it is also preferable that A of A-B in (I) is a peptide comprising MKIEEGKL sequence at the amino-terminus;

it is also preferable that A of A-B in (I) is one of peptides of the following SEQ ID NO: 64 to SEQ ID NO: 74, but is not limited thereto.

SEQ ID NO: 64:
MGSSHHHHHHSSGLVPRGSDMAGDNDDLDLEEALEPDMEEDDDQ
SEQ ID NO: 65:
MAGDNDDLDLEEALEPDMEEDDDQ
SEQ ID NO: 66:
MKIEEGKLAGDNDDLDLEEALEPDMEEDDDQ
SEQ ID NO: 67:
MSEQHAQGAGDNDDLDLEEALEPDMEEDDDQ
SEQ ID NO: 68:
MKIEEGKLAGDNDDLDLEEALEPDME
SEQ ID NO: 69:
MSEQHAQGAGDNDDLDLEEALEPDME
SEQ ID NO: 70:
MKIEEGKLEALEPDMEEDDDQ
SEQ ID NO: 71:
MSEQHAQGEALEPDMEEDDDQ
SEQ ID NO: 72:
MKIEEGKLAGDNDDLDLEEAL
SEQ ID NO: 73:
MSEQHAQGAGDNDDLDLEEAL
SEQ ID NO: 74:
MGSSHHHHHHSSAGDNDDLDLEEALEPDMEEDDDQ

In Embodiments given in this invention, it is preferable that the target protein B is PI (SEQ ID NO: 81), GCSF (SEQ ID NO: 82), GH (SEQ ID NO: 83) or BMP2 (SEQ ID NO: 84), but is not limited thereto.

The target proteins above mentioned in this invention also include their mutants, fragments and analogues with same functions.

As mentioned in the Embodiments, it is preferable that the PI is converted to insulin by the methods of enzymatic hydrolysis or chemical cleavage after be manufactured, but it is not limited to the specified methods.

Also this invention provides an expression vector which includes a gene encoding a fusion protein of A-B type wherein the fusion partner A is Px and the target protein B is PI, GH, GCSF, or BMP2; here x is 1, 2, 3, 4, 5, 6, 7, 8, 9, 10 or 11.

The invention includes microorganisms transformed with the above mentioned expression vector.

It is desirable that the transformants in this invention are E. coli strains BL21 (DE3), HMS174, or Rosetta DE3, and especially the E. coli Rosetta (DE3) (deposit number KCCM 1084P) is most preferable.

The invention is described as follows.

The present invention describes the technology to express recombinant proteins as a soluble form in E. coli, by providing a fusion protein of A-B type where the target protein B is linked to a fusion partner A. PI or insulin precursor, one of the target proteins, is expressed as a fusion protein, and the fusion PI or fusion insulin precursor is oxidized in the buffer solution resulting in correct disulfide bonds. The fusion PI or fusion insulin precursor is then enzymatically hydrolyzed to release the native and biologically active insulin. The beauty and revolution of this invention is to eliminate many cumbersome chemical steps such as solubilization of TB by denaturants, cyanogens bromide cleavage and sulfonation. Finally the invention produces insulin by reducing process steps from conventional 27 steps [Ladisch, M. R. (2001) In: Bioseparations Engineering pp. 520-521, Wiley-Interscience, N.Y. USA] to 12 steps.

This new approach and technology described in this invention are proven to effectively apply to any protein and all proteins including GH, GCSF, BMP-2 with no limitations.

### BRIEF DESCRIPTION OF THE DRAWINGS

FIG. 1 depicts Construction of plasmid pVEX-PxPI

FIG. 2 is SDS-PAGE result showing Px-proinsulin (PxPI) expression (M: Protein size markers, T: Total proteins, S: soluble part, P: insoluble part)

FIG. 3 is RP-HPLC analytic chart of enzymatic treatment of P1PI

FIG. 4 is comparison result between Insulin produced from P1PI by this invention and the Humulin, from Eli Lilly Company

FIG. 5 shows Mass spectrum of insulin produced from P1PI by this invention

FIG. 6 is SDS-PAGE result showing expression of P3hGCSF

FIG. 7 is SDS-PAGE result showing the purification of hGCSF and purified hGCSF after EKL cleavage

FIG. 8 is SDS-PAGE result showing expression of P3hGH

FIG. 9 is the purification of hGH and the P3hGH purification after EKL cleavage

FIG. 10 is SDS-PAGE result showing P1hBMP2 expression

FIG. 11 is SDS-PAGE result of P1hBMP2 purified through Ni-NTA column

FIG. 12 is SDS-PAGE result of hBMP2 after cleavage of P1hBMP2 by EKL

FIG. 13 shows Biological activity of hBMP2

### BEST MODES FOR CARRYING OUT THE INVENTION

Hereinafter, preferred Examples of the present invention will be described in detail with reference to the accompanying drawings. However, the description proposed herein is just a preferable example for the purpose of illustrations only, not intended to limit the scope of the invention, so it should be understood that other equivalents and modifications could be made thereto without departing from the spirit and scope of the invention.

### EXAMPLE 1

### PxPI Cloning

Fusion partners from P1PI to P11PI are composed of 25 or longer amino acid residues whose ratio of negative charge exceeds 30% and in which at least 5 out of 7 consecutive amino acid residues should contain negatively charged aspartic and/or glutamic acid residues. Two negative control groups are chosen for comparison to prove validity of this invention. One is fusion partners (P12 to P14) of the same size, but with reduced negative charge on the one hand, and the other fusion partners of the same charge (ratio of negative charge exceeds 30%), but with shorter peptide length (P15 to P17) of the fusion partners on the other hand.

### EXAMPLE 1-1

### P1PI Cloning

The molecular genetic technique used in the present invention is based on the literature [Ausubel, F. M. et al. (Ed.), J. Wiley Sons, Curr. Protocols in Molecular Biology, 1997]. The primers used for polymerase chain reaction (PCR) were custom synthesized at Bioneer Corp., rTag polymerase was purchased from TaKaRa, and PCR was carried out according to a standard condition presented by a TaKaRa's manual protocol.

Using human PI cDNA as a template, the gene encoding RR-PI (RRPI) sequence having two arginines (RR) at the amino terminus and restriction enzyme recognition sites, SalI at 5′-end and BamHI at 3′-end, respectively, was amplified by carrying out PCR. The sense primer (5′-GTC GAC CGT CGC TTC GTT AAT CAG CAC-3′, SEQ ID NO: 56) and antisense primer (5′-GGA TCC TCA GTT ACA ATA GTT-3′, SEQ ID NO: 57) were used for the PCR.

1 μg of amplified DNA fragment (SEQ ID NO: 18) was dissolved in 50 μl TE (pH 8.0) solution and mixed with 2 units of SalI (New England Biolabs) and 2 units of BamHI (New England Biolabs), and then the mixture were reacted for 16 hours at 37° C. to obtain a DNA fragment having restriction enzyme recognition sites, SalI at 5′-end and BamHI at 3′-end, respectively. In the same manner, a linear pT7-7 plasmid was prepared by treating circular pT7-7 plasmid with restriction enzymes SalI and BamHI, respectively. Subsequently, 20 ng of the DNA fragment and 20 ng of the linear pT7-7 plasmid were mixed in 10 μl TE (pH 8.0) solution, and then 1 unit of T4 DNA ligase was added to the mixture and reacted for 16 hours at 37° C. The plasmid so obtained was named pVEX-RRPI.

Subsequently, a DNA fragment comprising a base sequence encoding MGSSHHHHHHSSGLVPRGSDMAGDNDDLDLEEALEPDMEEDDDQ (SEQ ID NO: 64) for Px and restriction enzyme recognition sites, NdeI at 5′-end and SalI 3′-end, respectively, was amplified by PCR using sense primer (5′-CAT ATG GGC AGC AGC CAT CAT CAT CAT CAT CAC AGC AGC GGC CTG GTG CCG CGC GGC AGC GAC ATG GCG GGG GAC AAT GAC GAC CTC GAC CTG GAA GAA GCT SEQ ID NO: 22) and antisense primer (5′-GTC GAC CTG ATC GTC GTC TTC TTC CAT ATC TGG CTC TAA AGC TTC TTC-3′, SEQ ID NO: 23).

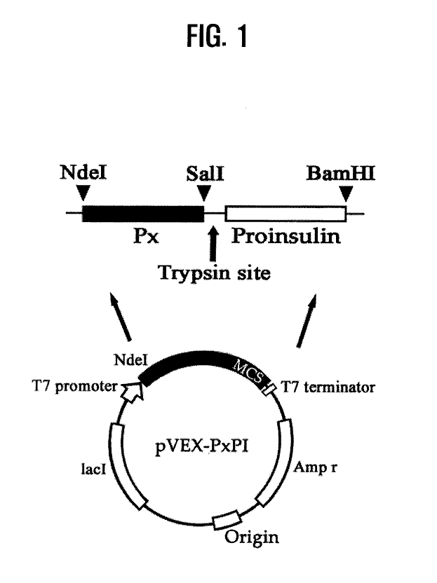

The amplified DNA fragment (SEQ ID NO: 1) was cleaved by restriction enzymes, Ndel and SalI and ligated with pVEX-RRPI which was prepared with the same restriction enzymes, and the resultant plasmid was named pVEX-P1PI (see FIG. 1).

### EXAMPLE 1-2

### P2PI Cloning

According to the same procedure described in Example 1-1, a DNA fragment containing a base sequence encoding MAGDNDDLDLEEALEPDMEEDDDQ (sequence number 65) for Px and restriction enzyme recognition sites, NdeI at 5′-end and SalI 3′-end, respectively, was amplified by PCR using sense primer (5′-CAT ATG GCG GGG GAC AAT GAC GAC CTC GAC CTG GAA GAA GCT-3′, sequence number 24) and antisense primer (5′-GTC GAC CTG ATC GTC GTC TTC TTC CAT ATC TGG CTC TAA AGC TTC TTC-3′, sequence number 25).

The amplified DNA fragment (SEQ ID NO: 2) was cleaved by restriction enzymes,

NdeI and SalI and ligated with pVEX-RRPI which was prepared with the same restriction enzymes, and the resultant plasmid was named pVEX-P2PI.

### Example 1-3

### P3PI Cloning

According to the same procedure described in Example 1-1, a DNA fragment comprising a base sequence encoding MKIEEGKLAGDNDDLDLEEALEPDMEEDDDQ (SEQ ID NO: 66) for Px and restriction enzyme recognition sites, NdeI at 5′-end and SalI 3′-end, respectively, was amplified by PCR using sense primer (5′-CAT ATG AAA ATC GAA GAA GGT AAA CTG GCG GGG GAC AAT GAC GAC CTC GAC CTG GAA GAA GCT TTA-3′, SEQ ID NO: 26) and antisense primer (5′-GTC GAC CTG ATC GTC GTC TTC TTC CAT ATC TGG CTC TAA AGC TTC TTC-3′, SEQ ID NO: 27).

The amplified DNA fragment (SEQ ID NO: 3) was cleaved by restriction enzymes, NdeI and SalI and ligated with pVEX-RRPI which was prepared with the same restriction enzymes, and the resultant plasmid was named pVEX-P3PI.

### EXAMPLE 1-4

### P4PI Cloning

According to the same procedure described in Example 1-1, a DNA fragment comprising a base sequence encoding MSEQHAQGAGDNDDLDLEEALEPDMEEDDDQ (SEQ ID NO: 67) for Px and restriction enzyme recognition sites, NdeI at 5′-end and SalI 3′-end, respectively, was amplified by PCR using sense primer (5′-CAT ATG TCT GAA CAA CAC GCA CAG GGC GCG GGG GAC AAT GAC GAC CTC GAC CTG GAA GAA GCT TTA-3′, SEQ ID NO: 28) and antisense primer (5′-GTC GAC CTG ATC GTC GTC TTC TTC CAT ATC TGG CTC TAA AGC TTC TTC-3′, SEQ ID NO: 29).

The amplified DNA fragment (SEQ ID NO: 4) was cleaved by restriction enzymes, NdeI and SalI and ligated with pVEX-RRPI which was prepared with the same restriction enzymes, and the resultant plasmid was named pVEX-P4PI.

### EXAMPLE 1-5

### P5PI Cloning

According to the same procedure described in Example 1-1, a DNA fragment comprising a base sequence encoding MKIEEGKLAGDNDDLDLEEALEPDME (SEQ ID NO: 68) for Px and restriction enzyme recognition sites, NdeI at 5′-end and SalI 3′-end, respectively, was amplified by PCR using sense primer (5′-CAT ATG AAA ATC GAA GAA GGT AAA CTG GCG GGG GAC AAT GAC GAC CTC GAC CTG GAA GAA GCT TTA-3′, SEQ ID NO: 30) and antisense primer (5′-GTC GAC TTC CAT ATC TGG CTC TAA AGC TTC TTC-3′, SEQ ID NO: 31).

The amplified DNA fragment (SEQ ID NO: 5) was cleaved by restriction enzymes, NdeI and SalI and ligated with pVEX-RRPI which was prepared with the same restriction enzymes, and the resultant plasmid was named pVEX-P5PI.

### EXAMPLE 1-6

### P6PI Cloning

According to the same procedure described in Example 1-1, a DNA fragment comprising a base sequence encoding MSEQHAQGAGDNDDLDLEEALEPDME (SEQ ID NO: 69) for Px and restriction enzyme recognition sites, NdeI at 5′-end and SalI 3′-end, respectively, was amplified by PCR using sense primer (5′-CAT ATG TCT GAA CAA CAC GCA CAG GGC GCG GGG GAC AAT GAC GAC CTC GAC CTG GAA GAA GCT TTA-3′, SEQ ID NO: 32) and antisense primer (5′-GTC GAC TTC CAT ATC TGG CTC TAA AGC TTC TTC-3′, SEQ ID NO: 33).

The amplified DNA fragment (SEQ ID NO: 6) was cleaved by restriction enzymes, NdeI and SalI and ligated with pVEX-RRPI which was prepared with the same restriction enzymes, and the resultant plasmid was named pVEX-P6PI.

### EXAMPLE 1-7

### P7PI Cloning

According to the same procedure described in Example 1-1, a DNA fragment comprising a base sequence encoding MKIEEGKLEALEPDMEEDDDQ (SEQ ID NO: 70) for Px and restriction enzyme recognition sites, NdeI at 5′-end and SalI 3′-end, respectively, was amplified by PCR using sense primer (5′-CAT ATG AAA ATC GAA GAA GGT AAA CTG GAA GCT TTA GAG CCA GAT-3′, SEQ ID NO: 34) and antisense primer (5′-GTC GAC CTG ATC GTC GTC TTC TTC CAT ATC TGG CTC-3′, SEQ ID NO: 35).

The amplified DNA fragment (SEQ ID NO: 7) was cleaved by restriction enzymes, NdeI and SalI and ligated with pVEX-RRPI which was prepared with the same restriction enzymes, and the resultant plasmid was named pVEX-P7PI.

### EXAMPLE 1-8

### P8PI Cloning

According to the same procedure described in Example 1-1, a DNA fragment comprising a base sequence encoding MSEQHAQGEALEPDMEEDDDQ (SEQ ID NO: 71) for Px and restriction enzyme recognition sites, NdeI at 5′-end and SalI 3′-end, respectively, was amplified by PCR using sense primer (5′-CAT ATG TCT GAA CAA CAC GCA CAG GGC GAA GCT TTA GAG CCA GAT-3, SEQ ID NO: 36) and antisense primer (5′-GTC GAC CTG ATC GTC GTC TTC TTC CAT ATC TGG CTC-3′, SEQ ID NO: 37).

The amplified DNA fragment (SEQ ID NO: 8) was cleaved by restriction enzymes, NdeI and SalI and ligated with pVEX-RRPI which was prepared with the same restriction enzymes, and the resultant plasmid was named pVEX-P8PI.

### Example 1-9

### P9PI Cloning

According to the same procedure described in Example 1-1, a DNA fragment comprising a base sequence encoding MMEEGKLAGDNDDLDLEEAL (SEQ ID NO: 72) for Px and restriction enzyme recognition sites, NdeI at 5′-end and SalI 3′-end, respectively, was amplified by PCR using sense primer (5′-CAT ATG AAA ATC GAA GAA GGT AAA CTG GCG GGG GAC AAT GAC GAC CTC GAC CTG GAA-3′, SEQ ID NO: 38) and antisense primer (5′-GTC GAC TAA AGC TTC TTC CAG-3′, SEQ ID NO: 39).

The amplified DNA fragment (SEQ ID NO: 9) was cleaved by restriction enzymes, NdeI and SalI and ligated with pVEX-RRPI which was prepared with the same restriction enzymes, and the resultant plasmid was named pVEX-P9PI.

### EXAMPLE 1-10

### P10PI Cloning

According to the same procedure described in Example 1-1, a DNA fragment comprising a base sequence encoding MSEQHAQGAGDNDDLDLEEAL (s SEQ ID NO: 73) for Px and restriction enzyme recognition sites, NdeI at 5′-end and SalI 3′-end, respectively, was amplified by PCR using sense primer (5′-CAT ATG TCT GAA CAA CAC GCA CAG GGC GCG GGG GAC AAT GAC GAC CTC GAC CTG GAA-3′, SEQ ID NO: 40) and antisense primer (5′-GTC GAC TAA AGC TTC TTC CAG-3′, SEQ ID NO: 41).

The amplified DNA fragment (SEQ ID NO: 10) was cleaved by restriction enzymes, NdeI and SalI and ligated with pVEX-RRPI which was prepared with the same restriction enzymes, and the resultant plasmid was named pVEX-P10PI.

### EXAMPLE 1-11

### P11PI Cloning

According to the same procedure described in Example 1-1, a DNA fragment comprising a base sequence encoding MGSSHHHHHHSSAGDNDDLDLEEALEPDMEEDDDQ (SEQ ID NO: 74) for Px and restriction enzyme recognition sites, NdeI at 5′-end and SalI 3′-end, respectively, was amplified by PCR using sense primer (5′-CAT ATG GGC AGC AGC CAT CAT CAT CAT CAT CAC AGC AGC GCG GGG GAC AAT GAC GAC CTC GAC CTG GAA GAA GCT-3′, SEQ ID NO: 42) and antisense primer (5′-GTC GAC CTG ATC GTC GTC TTC TTC CAT ATC TGG CTC TAA AGC TTC TTC-3′, SEQ ID NO: 43).

The amplified DNA fragment (SEQ ID NO: 11) was cleaved by restriction enzymes, NdeI and SalI and ligated with pVEX-RRPI which was prepared with the same restriction enzymes, and the resultant plasmid was named pVEX-P11PI.

### EXAMPLE 1-12

### P12PI Cloning

According to the same procedure described in Example 1-1, a DNA fragment comprising a base sequence encoding MKIEEGKLAGDNVLLDLILALAPIME (SEQ ID NO: 75) for Px and restriction enzyme recognition sites, NdeI at 5′-end and SalI 3′-end, respectively, was amplified by PCR using sense primer (5′-CAT ATG AAA ATC GAA GAA GGT AAA CTG GCG GGG GAC AAT GTC CTC CTC GAC CTG ATC TTA GCT TTA GCG-3′, SEQ ID NO: 44) and antisense primer (5′-GTC GAC TTC CAT AAT TGG CGC TAA AGC TAA-3′, SEQ ID NO: 45).

The amplified DNA fragment (SEQ ID NO: 12) was cleaved by restriction enzymes, NdeI and SalI and ligated with pVEX-RRPI which was prepared with the same restriction enzymes, and the resultant plasmid was named pVEX-P12PI.

### EXAMPLE 1-13

### P13PI Cloning

According to the same procedure described in Example 1-1, a DNA fragment comprising a base sequence encoding MKIEEGKLEALVPIMVADVAQ (SEQ ID NO: 76) for Px and restriction enzyme recognition sites, NdeI at 5′-end and SalI 3′-end, respectively, was amplified by PCR using sense primer (5′-CAT ATG AAA ATC GAA GAA GGT AAA CTG GAA GCT TTA GTG CCA ATT ATG GTA GCA GAC-3′, SEQ ID NO: 46) and antisense primer (5′-GTC GAC CTG AGC GAC GTC TGC TAC CAT AAT-3′, SEQ ID NO: 47).

The amplified DNA fragment (SEQ ID NO: 13) was cleaved by restriction enzymes, NdeI and SalI and ligated with pVEX-RRPI which was prepared with the same restriction enzymes, and the resultant plasmid was named pVEX-P13PI.

### Example 1-14

### P14PI Cloning

According to the same procedure described in Example 1-1, a DNA fragment comprising a base sequence encoding MKIEEGKLAGDNVLLDLILAL (SEQ ID NO: 77) for Px and restriction enzyme recognition sites, NdeI at 5′-end and SalI 3′-end, respectively, was amplified by PCR using sense primer (5′-CAT ATG AAA ATC GAA GAA GGT AAA CTG GCG GGG GAC AAT GTC CTC CTC GAC CTG ATC-3′, SEQ ID NO: 48) and antisense primer (5′-GTC GAC TAA AGC TAA GAT CAG-3′, SEQ ID NO: 49).

The amplified DNA fragment (SEQ ID NO: 14) was cleaved by restriction enzymes, NdeI and SalI and ligated with pVEX-RRPI which was prepared with the same restriction enzymes, and the resultant plasmid was named pVEX-P14PI.

### Example 1-15

### P15PI Cloning

According to the same procedure described in Example 1-1, a DNA fragment comprising a base sequence encoding MKIEEGKLEALEPDMEE (SEQ ID NO: 78) for Px and restriction enzyme recognition sites, NdeI at 5′-end and SalI 3′-end, respectively, was amplified by PCR using sense primer (5′-CAT ATG AAA ATC GAA GAA GGT AAA CTG GAA GCT TTA GAG CCA GAT-3′, SEQ ID NO: 50) and antisense primer (5′-GTC GAC TTC TTC CAT ATC TGG CTC TAA-3′, SEQ ID NO: 51).

The amplified DNA fragment (SEQ ID NO: 15) was cleaved by restriction enzymes, NdeI and SalI and ligated with pVEX-RRPI which was prepared with the same restriction enzymes, and the resultant plasmid was named pVEX-P15PI.

### EXAMPLE 1-16

### P16PI Cloning

According to the same procedure described in Example 1-1, a DNA fragment comprising a base sequence encoding MKIEEGKLAGDNDDLDLE (SEQ ID NO: 79) for Px and restriction enzyme recognition sites, NdeI at 5′-end and SalI 3′-end, respectively, was amplified by PCR using sense primer (5′-CAT ATG AAA ATC GAA GAA GGT AAA CTG GCG GGG GAC AAT GAC GAC CTC-3′, SEQ ID NO: 52) and antisense primer (5′-GTC GAC TTC CAG GTC GAG GTC GTC-3′, SEQ ID NO: 53).

The amplified DNA fragment (SEQ ID NO: 16) was cleaved by restriction enzymes, NdeI and SalI and ligated with pVEX-RRPI which was prepared with the same restriction enzymes, and the resultant plasmid was named pVEX-P16PI.

### EXAMPLE 1-17

### P17PI Cloning

According to the same procedure described in Example 1-1, a DNA fragment comprising a base sequence encoding MSEQHAQGAGDNDDLDLE (SEQ ID NO: 80) for Px and restriction enzyme recognition sites, NdeI at 5′-end and SalI 3′-end, respectively, was amplified by PCR using sense primer (5′-CAT ATG TCT GAA CAA CAC GCA CAG GGC GCG GGG GAC AAT GAC GAC CTC-3′, SEQ ID NO: 54) and antisense primer (5′-GTC GAC TTC CAG GTC GAG GTC GTC-3′, SEQ ID NO: 55).

The amplified DNA fragment (SEQ ID NO: 17) was cleaved by restriction enzymes, NdeI and SalI and ligated with pVEX-RRPI which was prepared with the same restriction enzymes, and the resultant plasmid was named pVEX-P17PI.

### EXAMPLE 2

### Preparation of E. Coli Transformants

BL21 (DE3), HMS 174 (DE3) or Rosseta (DE3), representative strains of E. coli were transformed respectively with one of the expression plasmids pVEX-PxPI, prepared in Example 1, and the ampicillin-resistant colonies were selected [Hanahan, D. (1985) DNA Cloning vol. 1 (Ed. D. M. Glover) 109-135, IRS press].

The strain E. coli Rosetta (DE3) transformed with the expression vector pSSU-P3PI (same as pVEX-P3PI) were selected and deposited in an international depository authority, the Korean Culture Center of Microorganisms (KCCM, #361-221 Yurim Building, Hongje-1-dong, Seodaemun-gu, Seoul, Republic of Korea) on Oct. 12, 2005 under an accession number of KCCM-10684P according to the Budapest Convention.

### Example 3

### Culture of E. Coli Transformants and PxPI Expression

The E. coli strain transformed with the recombinant expression vector pVEX-PxPI of the above mentioned Example 1 was inoculated and cultured in LB liquid culture medium (tryptone 10 g, yeast extract 10 g, and NaCl 5 g in 1 liter) containing ampicillin (50˜100 μg/ml) or ampicillin and chloramphenicol (38˜50 μg/ml each).

The recombinant E. coli strains were cultured in the solid medium containing the same components as in the liquid medium, and the resultant colonies were cultured for 12 hours in 1 ml of a liquid medium containing ampicillin (50˜100 μg/ml) or ampicillin and chloramphenicol (38˜50 μg/ml), and then the culture broth was suspended in 15% glycerol and kept at −70° C. for future use.

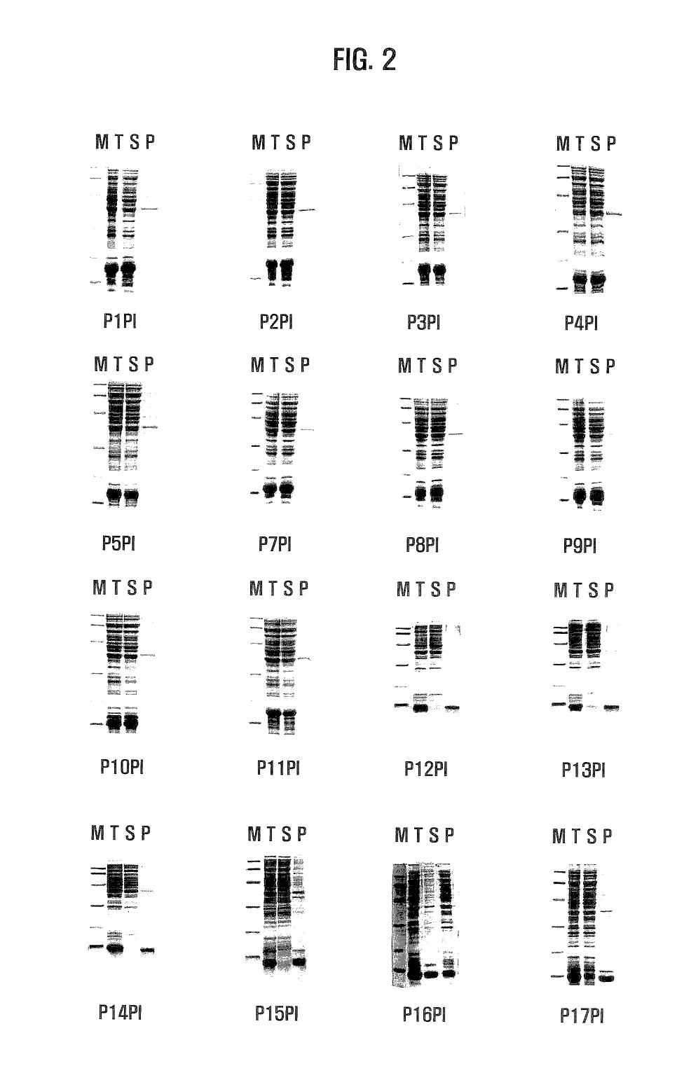

The recombinant E. coli strains stored at −70° C. were spread on the solid culture medium of the same composition as in above and cultured at 37° C. for 16˜18 hours. The resulting colonies were inoculated to 20 ml of liquid medium and cultured at 37° C. while stirring at a rotary speed of 200 rpm. Upon completion of cultivation for 16-17 hours the resultant culture broth was inoculated to 400 ml of liquid medium and cultured at 37° C., pH 7.0 while stirring at a rotary speed of 200 rpm. When the recombinant E. coli strains were grown to an optical density of 0.4˜0.6 at 600 nm, isopropyl-β-D-thiogalactopyranoside (IPTG) was added to the culture broth to a final concentration of 0.5-1.0 mM, and the culture broth was further cultured for 4 hours at 20˜25° C. while stirring at a rotary speed of 200 rpm to induce expression of fusion proteins. The resultant culture broth was centrifuged for 10 min at 6000 rpm to obtain E. coli pellets, and the pellets were suspended in 20 ml of 50 mM Tris buffer and 50 mM glycine buffer solution (pH 8.0˜10.0) and then lysed by ultrasonication. The cell lysates were centrifuged at 13,000 rpm for 10 minutes at 4° C. to separate supernatant from pellet, and then the amounts of fusion proteins partitioned in soluble and insoluble fractions were determined on SDS-PAGE. As a result, a majority of the PxPI fusion proteins were overexpressed in a soluble form, while most of negative control group were expressed as insoluble precipitate (see FIG. 2 and Table 1).

TABLE 1

| | | | Ratio of | | |
|---|---|---|---|---|---|
| | | Length of Px | negative | Expression rate | |
| | | (number of | charges | at 37° C. (%) | |
| Proteins | | amino acids) | in Px (%) | soluble | insoluble |
| P1PI | | 48 | 31 | >95 | <5 |
| P2PI | | 28 | 50 | >95 | <5 |
| P3PI | | 35 | 46 | >95 | <5 |
| P4PI | | 35 | 43 | >95 | <5 |
| P5PI | | 30 | 40 | >95 | <5 |
| P7PI | | 25 | 44 | >95 | <5 |
| P8PI | | 25 | 40 | >95 | <5 |
| P9PI | | 25 | 36 | >95 | <5 |
| P10PI | | 25 | 32 | >95 | <5 |
| P11PI | | 39 | 36 | >95 | <5 |
| Negative | P12PI | 30 | 20 | 18 | 82 |
| control | P13PI | 25 | 20 | 25 | 82 |
| group | P14PI | 25 | 20 | 20 | 80 |
| | P15PI | 21 | 38 | 10 | 90 |
| | P16PI | 22 | 36 | 43 | 56 |
| | P17PI | 22 | 36 | 5 | 95 |

### EXAMPLE 4

### Insulin Production from PxPI

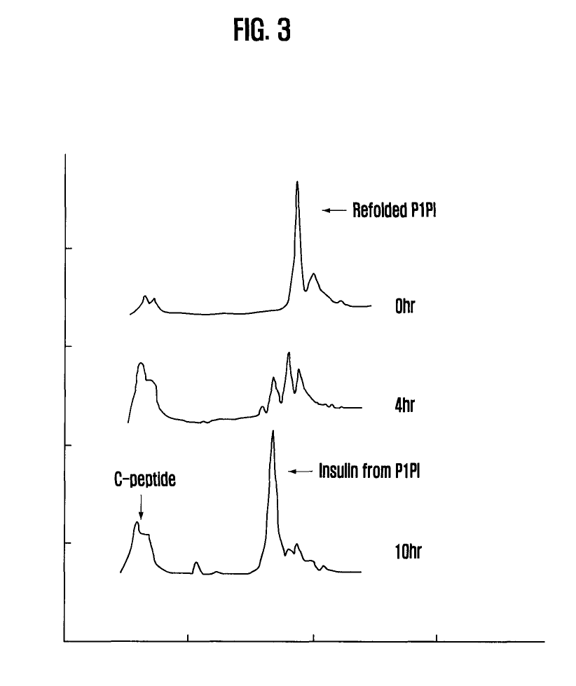

The lysed E. coli cell suspension prepared by ultrasonication in Example 3 was centrifuged at 13,000 rpm, for 10 minutes at 4° C. to separate the supernatant and the pellet. After cysteine and cystine (0˜3 mM of cysteine and 1-10 mM of cystine) were added to the supernatant and the solution was allowed to react for 15 hours at room temperature, trypsin and carboxypeptidase B were added to the solution so that the final concentration maintains at the ratio of PxPI:trypsin=500:1 and PxPI:carboxypeptidase B=300:1. The pH of the solution was adjusted to pH 8.0 and the reaction was carried out at 15° C. During the enzymatic treatment, the sample was taken at a given interval and analyzed by the reverse phase HPLC using analytical C8 column at 280 nm (FIG. 3).

### EXAMPLE 5

### Comparison Between the Sample Insulin Made from PxPI and Commercial Insulin

The sample insulin purified in Example 4 was compared with the commercial insulin (humulin).

### EXAMPLE 5-1

### Reverse Phase HPLC and Mass Spectroscopy

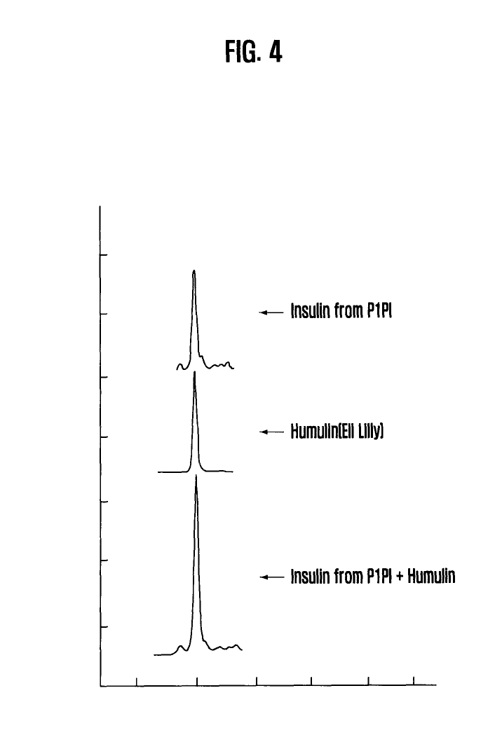

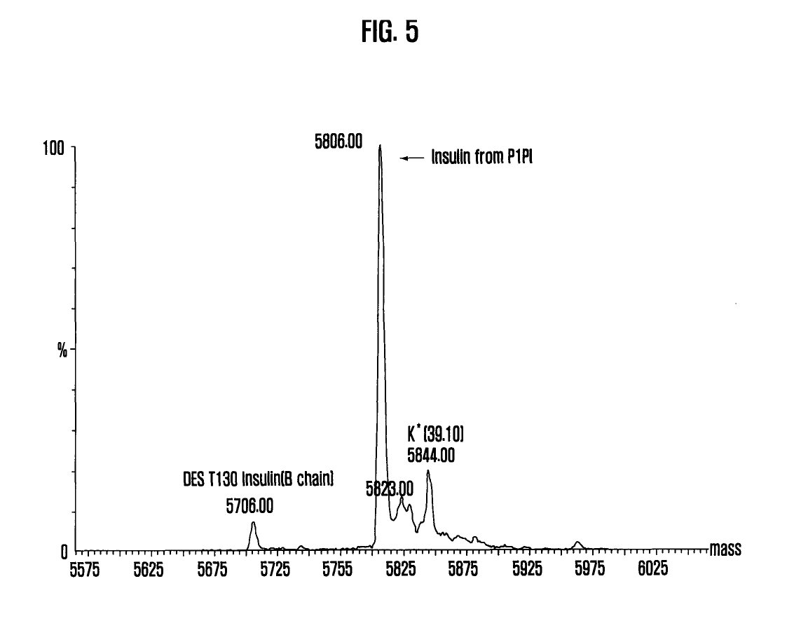

It was confirmed that the sample insulin prepared by this invention is identical to the commercial insulin (humulin) by the reverse phase HPLC using analytical C8 column (FIG. 4). According to the mass analysis, the sample insulin prepared by this invention has molecular weight of 5806.43±0.6 Da, which is identical to the theoretical value of 5807.19 Da within the error range (FIG. 5).

### EXAMPLE 5-2

### Measurement of Insulin Activity

In order to measure the insulin activity prepared in Example 4, 8 weeks-old male Sprague-Dawley (SD) rats with 200˜250 g body weight were given a subcutaneous injection of 0.1 ml/100 g of body weight of insulin which was dissolved in phosphate buffer (8 g NaCl, 0.2 g KCl, 1.44 g Na₂HPO₄ and 0.24 g NaH₂PO₄) at a concentration of 4˜80 μg/0.1 ml. Blood samples were taken from the tail vein at the interval of 30 min., 1, 2, 3, and 4 hours, and the hypoglycemic effects, presented as the ED₅₀ values, were determined (Table 2). The ED₅₀ values represent the dose of the insulin that gives half the maximum hypoglycemic activity at 1 or 2 hours after subcutaneous administration.

TABLE 2

| Insulin | 1 hour | 2 hours |
|---|---|---|
| Humulin (Eli Lilly) | 1.5 ± 0.34 | 1.8 ± 0.37 |
| Sample insulin of this invention | 1.5 ± 0.37 | 1.8 ± 0.25 |

Table 2 demonstrates hypoglycemic activity of insulins(nmol/kg)

### EXAMPLE 6

### Application of the Invention to the Expression of Other Proteins as a Soluble Form

In order to demonstrate the effectiveness of fusion partners in this invention for the expression of various other proteins as a soluble form, the present technology was applied to the production of human granulocyte colony stimulating factor (hGCSF), human growth hormone (hGH) and human bone morphogenetic protein 2 (hBMP2), etc.

### EXAMPLE 6-1

### Expression and Purification of hGCSF

### EXAMPLE 6-1-1

### Cloning and Expression of P3hGCSF

Using human GCSF cDNA as a template, the DNA fragment encoding hGCSF sequence and restriction enzyme recognition sites, SalI at 5′-end and BamHI at 3′-end, respectively, was amplified by carrying out PCR. The sense primer (5′-GTC GAC GAC GAC GAC AAA ACC CCC CTG-3′, SEQ ID NO: 58) and antisense primer (5′-GGA TCC TCA GGG CTG GGC AAG-3′, SEQ ID NO: 59) were used for the PCR. Amplified hGCSF DNA (SEQ ID NO: 19) was treated with SalI and BamHI, respectively, and inserted into the expression vector pVEX-P3PI treated with the same restriction enzymes. The resulting plasmid (pVEX-P3hGCSF) was then used to transform E. coli Rosetta (DE3).

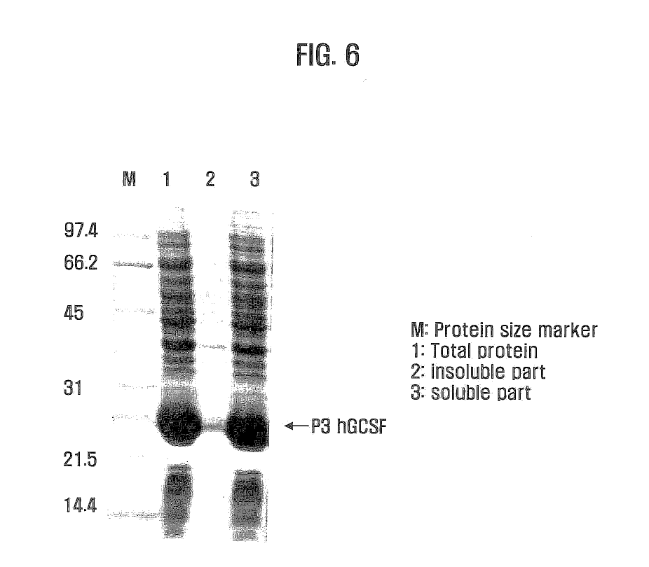

The E. coli Rosetta (DE3) transformant was induced to express P3hGCF in the same manner as described in Example 3. Separation of the soluble and insoluble parts by centrifugation and subsequent SDS-PAGE protein analysis confirmed that P3hGCSF was exclusively expressed in a soluble form (FIG. 6).

### EXAMPLE 6-1-2

### Purification of hGCSF

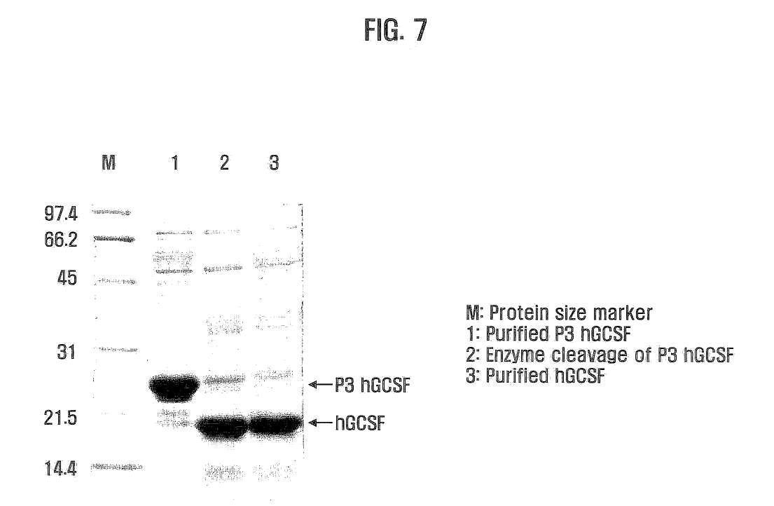

The soluble part of expressed P3hGCSF was purified on Q-Sepharose anion-exchange column (GE Healthcare Bioscience) using 30 mM Tris buffer, pH 8.0 with a linear gradient of 0˜1 M NaCl. Enterokinase (EKL) was added to the purified P3hGCSF to a final ratio of P3hGSCF:EKL=50:1, and the mixture was reacted for 24 hours at 37° C. to isolate hGCSF. The isolated hGCSF was then purified on a Q-Sepharose anionic exchange column in a manner same as above. SDS-PAGE analysis confirmed the purity of hGCSF (FIG. 7).

Overall production yield starting from LB liquid culture to the final Q-Sepharose anion-exchange chromatography was about 30% and ˜8 mg of hGCSF was obtained from 1 liter of LB culture.

### EXAMPLE 6-1-3

### N-Terminal Sequence Analysis of Purified hGCSF

The purified hGCSF was blotted on PVDF membrane and N-terminal sequence analysis was carried out using Milligen 6600B. PHT-amino acid derivatives were produced by using Edman degradation method and analyzed on RP-HPLC. Based on the sequence analysis, the purified hGCSF has a sequence corresponding to NH₂-Thr-Pro-Leu-Gly-Pro, which is identical to the physiologically active human GCSF.

### EXAMPLE 6-2

### Expression and Activity Measurement of hGH

### EXAMPLE 6-2-1

### Cloning and Expression of P3hGH

Using hGH cDNA as a template, the DNA fragment encoding hGH sequence and restriction enzyme recognition sites, SalI at 5′-end and BamHI at 3′-end, respectively, was amplified by carrying out PCR. The sense primer (5′-GTC GAC GAC GAC GAC AAA TTC CCA ACC NIT CCC-3′, SEQ ID NO: 60) and antisense primer (5′-GGA TCC TCA GAA GCC ACA GCT GCC-3′, SEQ ID NO: 61) were used for the PCR. Amplified hGH DNA (SEQ ID NO: 20) was treated with SalI and BamHI, respectively, and inserted into the expression vector pVEX-P3PI treated with the same restriction enzymes. The resulting plasmid (pVEX-P3hGH) was then used to transform E. coli Rosetta (DE3).

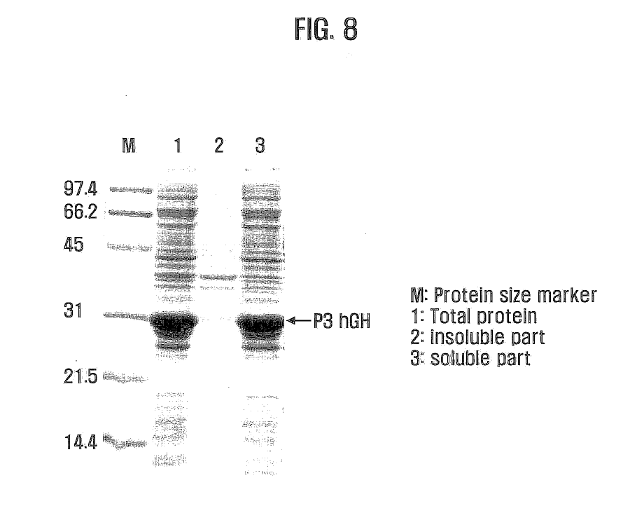

The E. coli Rosetta (DE3) transformant was induced to express P3hGH in the same manner as described in Example 3. Separation of the soluble and insoluble parts by centrifugation and subsequent SDS-PAGE protein analysis confirmed that P3hGH was exclusively expressed in a soluble form (FIG. 8).

### EXAMPLE 6-2-2

### Purification of hGH

According to the same procedure described in Example 6-1-2, the soluble part of expressed P3hGH was purified on Q-Sepharose anion-exchange column (GE Healthcare Bioscience) using 30 mM Tris buffer, pH 8.0 with a linear gradient of 0.2˜0.75 M NaCl. The hGH was isolated from the purified P3hGH.

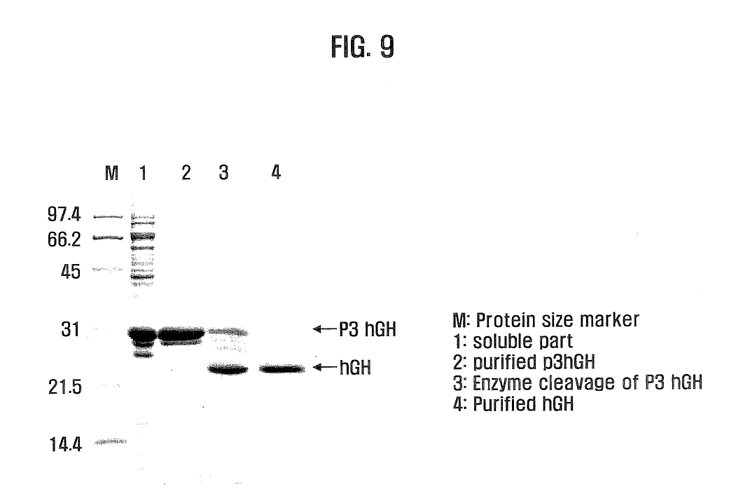

According to the same procedure described in Example 6-1-2, The hGH was isolated from fusion partner by treating the purified P3hGH with EKL. The isolated hGH was then purified on a Q-Sepharose anionic exchange column in a manner same as above (FIG. 9).

### EXAMPLE 6-2-3

### Activity Measurement of hGH

The activity of the purified hGH in Example 6-2-2 was measured using radioreceptor analysis (Journal of Korean Endocrinology Society 5(3), 1990) and revealed that the activity is 2.60 IU/mg, which is slightly higher than 2.5 IU/mg of hGH (NBSB 80/5050) derived from human pituitary grand which was supplied by WHO.

### EXAMPLE 6-3

### Expression and Activity Measurement of hBMP2

### EXAMPLE 6-3-1

### Cloning and Expression of hBMP2

Using hBMP2 cDNA as a template, the DNA fragment encoding hBMP2 sequence and restriction enzyme recognition sites, SalI at 5′-end and BamHI at 3′-end, respectively, was amplified by carrying out PCR. The sense primer (5′-GTC GAC GAC GAC GAC AAG CAA GCC AAA CAC AAA-3′, SEQ ID NO: 62) and antisense primer (5′-GGA TCC TCA GCG ACA CCC ACA ACC-3′, SEQ ID NO: 63) were used for the PCR. Amplified hBMP2 DNA (SEQ ID NO: 21) was treated with SalI and BamHI, respectively, and inserted into the expression vector pVEX-P3PI treated with the same restriction enzymes. The resulting plasmid (pVEX-P3hBMP2) was then used to transform E. coli Rosetta (DE3).

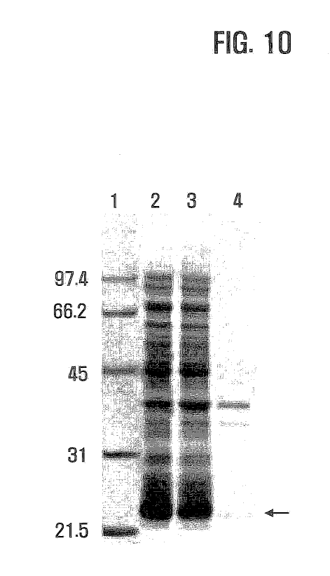

The E. coli Rosetta (DE3) transformant was induced to express P3hBMP2 in the same manner as described in Example 3. Separation of the soluble and insoluble parts by centrifugation and subsequent SDS-PAGE protein analysis confirmed that P3hBMP2 was exclusively expressed in a soluble form (FIG. 10).

### EXAMPLE 6-3-2

### Purification of hBMP2

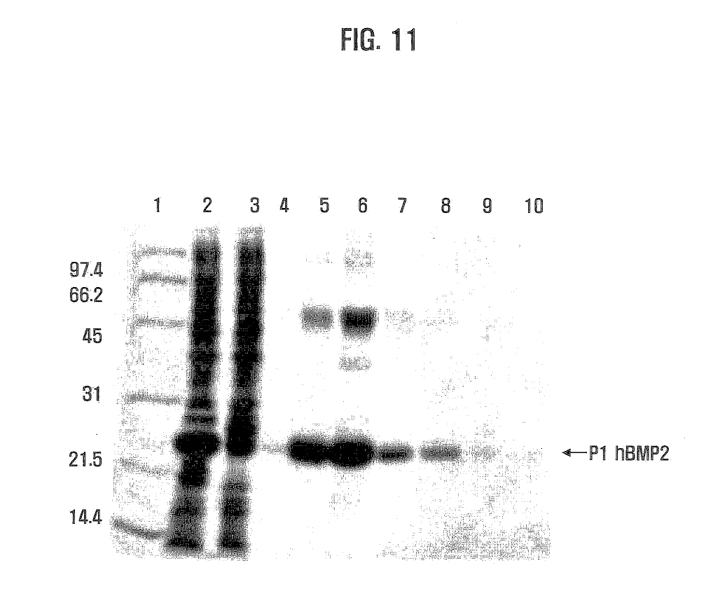

The soluble part of the expressed P1hBMP2 was purified on Nickel-NTA agarose column using 50 mM Tris buffer, pH 8.0 with a linear gradient of 20˜400 mM imidazole (FIG. 11).

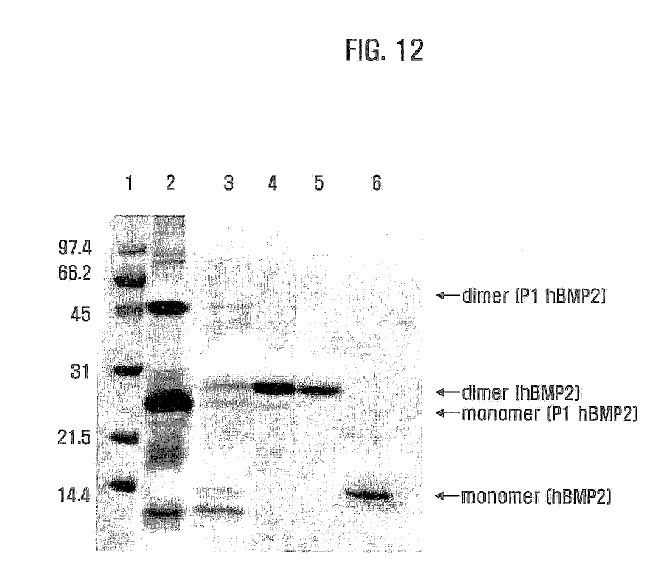

The hBMP2 was isolated from the purified P3hBMP2 using EKL following the method as described in Example 6-1-2. The isolated hBMP2 was then purified on a heparin column using 20 mM Tris buffer, 4M urea (pH 8.5) with a linear gradient of 0˜1 M NaCl (FIG. 12).

### EXAMPLE 6-3-3

### Activity Measurement of hBMP2

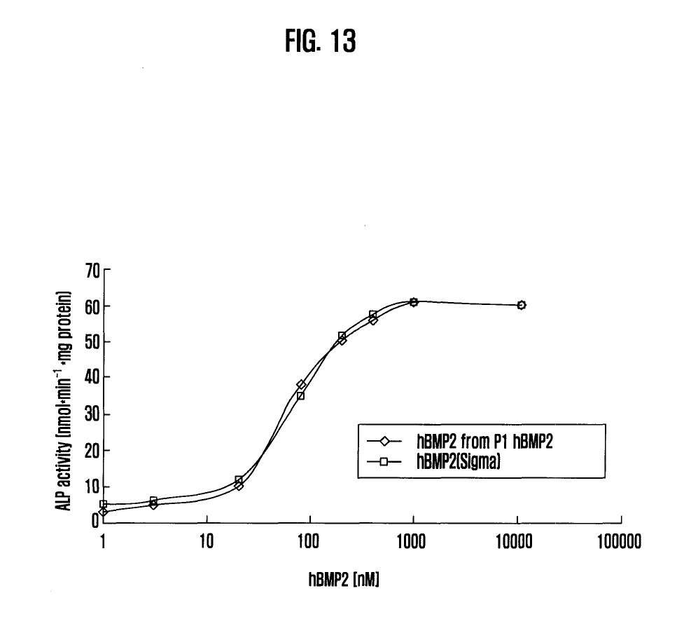

The hBMP2 activity was analyzed by the method of alkaline phosphatase induction [Katagiri et al. (1990) Biochem. Biophys. Res. Commun. 172, 295-299]. Mouse fibroblast cells from the line C3H10T1/2 in BME-Earle medium plus 10% fetal calf serum were incubated at 1×10⁵ cells/ml in 1-ml aliquots in a 24-well plate for 24 hours at 37° C. and 10% CO₂. After removal of the supernatant, 1 ml fresh medium was added with various concentrations of hBMP-2. After a further cultivation for 4 days, cells were lysed in 0.2 ml buffer (0.1 M glycerol, pH 9.6, 1% NP-40, 1 mM MgCl₂, 1 mM ZnCl₂) and alkaline phosphatase activity was determined using 150 μl 0.3 mM p-nitrophenylphosphate in the pH 9.6 buffer as substrate. Absorbance at 405 nm was recorded after 20 min incubation at 37° C. The results confirmed that the activity of hBMP2 prepared by this invention is identical to the standard hBMP2 (FIG. 13).

### INDUSTRIAL APPLICABILITY

As described above, the present invention provides a new technology to dramatically reduce the process steps involved in the production of human insulin by inhibiting the formation of IB and to convert fusion PI to insulin in a single step. The plasmids and the method of insulin production using the plasmids in this invention warrant high production yield by minimizing unwanted byproducts, production steps and cost.

As a result, the plasmids and the method of producing polypeptides made by this invention can be effectively applied to the mass production of human insulin in industrial scale. The technology provided by this invention can also be applied to the production of other proteins including GH, GCSF and BMP2.

What is claimed is:

1. A method for preparing a polypeptide using microorganism transformed by a gene encoding a fusion protein of the A-B type:
A-B (I)
wherein A is a fusion partner comprises 25 or more amino acid residues comprising aspartic acid and glutamic acid residues in which total negative charges of the fusion partner exceeds 30%, and B is a target protein to be produced.

2. The method for preparing a polypeptide according to claim 1, wherein part of the fusion partner A comprises a sequence of 7 consecutive amino acid residues with 5 or more negative charges.

3. The method according to claim 1, wherein the fusion partner A is a peptide comprising MKIEEGKL at the amino terminus.

4. The method for preparing a polypeptide according to claim 1, wherein the fusion peptide A is a peptide comprising one of a SEQ ID NO: 64 to 74 listed below.
SEQ ID NO: 64:
MGSSHHHHHHSSGLVPRGSDMAGDNDDLDLEEALEPDMEEDDDQ
SEQ ID NO: 65:
MAGDNDDLDLEEALEPDMEEDDDQ
SEQ ID NO: 66:
MKIEEGKLAGDNDDLDLEEALEPDMEEDDDQ
SEQ ID NO: 67:
MSEQHAQGAGDNDDLDLEEALEPDMEEDDDQ
SEQ ID NO: 68:
MKIEEGKLAGDNDDLDLEEALEPDME
SEQ ID NO: 69:
MSEQHAQGAGDNDDLDLEEALEPDME
SEQ ID NO: 70:
MKIEEGKLEALEPDMEEDDDQ
SEQ ID NO: 71:
MSEQHAQGEALEPDMEEDDDQ
SEQ ID NO: 72:
MKIEEGKLAGDNDDLDLEEAL
SEQ ID NO: 73:
MSEQHAQGAGDNDDLDLEEAL
SEQ ID NO: 74:
MGSSHHHHHHSSAGDNDDLDLEEALEPDMEEDDDQ

5. The method for preparing a pro insulin or its analogues according to claims 1.

6. The method for preparing insulin, by further comprising the process of enzymatic or chemical cleavage after producing the proinsulin or its analogues according to claim 5.

7. (canceled)

8. A method for preparing a Granulocyte colony stimulating factor (GCSF), or its analogues according to claims 1.

9. (canceled)

10. A method for preparing a Growth hormone (GH) or its analogues according to claims 1.

11. (canceled)

12. A method for preparing a Bone morphogenetic protein (BMP)-2 or its analogues according to claims 1.

13. (canceled)

14. The method for preparing a polypeptide according to claims 1, wherein the method further comprises obtaining the target protein from the fusion protein by incorporating enzymatic or chemical cleavage site to the carboxyl-terminus of the fusion partner.

15. A fusion protein of A-B type;
A-B (I)
wherein A is a fusion partner comprising 25 or more amino acid residues comprising aspartic acid and glutamic acid residues in which total negative charges of the fusion partner exceeds 30%, and B is a target protein to be produced.

16. The fusion protein according to claim 15, wherein the fusion partner A is a peptide comprising a sequence of 7 consecutive amino acid residues with 5 or more negative charges;

17. The fusion protein according to claim 15, wherein the fusion partner A is a peptide comprising MKIEEGKL at the amino terminus.

18. The fusion protein according to claim 15, wherein the fusion peptide A is a peptide comprising one of a SEQ 10 NO: 64-74 listed below.
SEQ ID NO: 64:
MGSSHHHHHHSSGLVPRGSDMAGDNDDLDLEEALEPDMEEDDDQ
SEQ ID NO: 65:
MAGDNDDLDLEEALEPDMEEDDDQ
SEQ ID NO: 66:
MKIEEGKLAGDNDDLDLEEALEPDMEEDDDQ
SEQ ID NO: 67:
MSEQHAQGAGDNDDLDLEEALEPDMEEDDDQ
SEQ ID NO: 68:
MKIEEGKLAGDNDDLDLEEALEPDME
SEQ ID NO: 69:
MSEQHAQGAGDNDDLDLEEALEPDME
SEQ ID NO: 70:
MKIEEGKLEALEPDMEEDDDQ
SEQ ID NO: 71:
MSEQHAQGEALEPDMEEDDDQ
SEQ ID NO: 72:
MKIEEGKLAGDNDDLDLEEAL
SEQ ID NO: 73:
MSEQHAQGAGDNDDLDLEEAL
SEQ ID NO: 74:
MGSSHHHHHHSSAGDNDDLDLEEALEPDMEEDDDQ

19. The fusion protein according to claim 15, wherein the target protein B is a Proinsulin, a Growth Hormone, a Granulocyte colony stimulating factor, or a Bone morphogenetic protein-2.

20. The fusion protein according to claim 19, wherein the proinsulin is converted to insulin by enzymatic or chemical cleavage.

21. An expression vector which comprises a gene encoding a fusion protein of A-B type wherein the fusion partner A is Px and the target protein B is a Pro insulin, a Growth Hormone, a Granulocyte colony stimulating factor, or a Bone morphogenetic protein-2; wherein x is 1, 2, 3, 4, 5, 6, 7, 8, 9, 10 or 11.

22. A microorganism transformed with the expression vector in claim 21.

23. The microorganism according to claim 22, wherein the microorganism is E. coli BL21 (DE3), HMS174 (DE3) or Rosetta (DE3).

24. The microorganism according to claim 23, wherein the microorganism is E. coli Rosetta (DE3) (Accession number KCCM 10684P).


SEQUENCE LISTING (84 sequences)

SEQ ID NO: 1 - DNA, 147 bp, Artificial Sequence
  Human proinsulin and fusion partner
catatgggcagcagccatcatcatcatcatcacagcagcggcctggtgccgcgcggcagcgacatggcgggggacaatgacgacctcgacctggaagaagctttagagccagatatggaagaagacgacgatcaggtcgacgtcgac

SEQ ID NO: 2 - DNA, 81 bp, Artificial Sequence
  Human proinsulin and fusion partner
catatggcgggggacaatgacgacctcgacctggaagaagctttagagccagatatggaagaagacgacgatcaggtcgac

SEQ ID NO: 3 - DNA, 102 bp, Artificial Sequence
  Human proinsulin and fusion partner
catatgaaaatcgaagaaggtaaactggcgggggacaatgacgacctcgacctggaagaagctttagagccagatatggaagaagacgacgatcaggtcgac

SEQ ID NO: 4 - DNA, 108 bp, Artificial Sequence
  Human proinsulin and fusion partner
catatgtctgaacaacacgcacagggcgcgggggacaatgacgacctcgacctggaagaagctttagagccagatatggaagaagacgacgatcaggtcgacgtcgac

SEQ ID NO: 5 - DNA, 87 bp, Artificial Sequence
  Human proinsulin and fusion partner
catatgaaaatcgaagaaggtaaactggcgggggacaatgacgacctcgacctggaagaagctttagagccagatatggaagtcgac

SEQ ID NO: 6 - DNA, 87 bp, Artificial Sequence
  Human proinsulin and fusion partner
catatgtctgaacaacacgcacagggcgcgggggacaatgacgacctcgacctggaagaagctttagagccagatatggaagtcgac

SEQ ID NO: 7 - DNA, 72 bp, Artificial Sequence
  Human proinsulin and fusion partner
catatgaaaatcgaagaaggtaaactggaagctttagagccagatatggaagaagacgacgatcaggtcgac

SEQ ID NO: 8 - DNA, 72 bp, Artificial Sequence
  Human proinsulin and fusion partner
catatgtctgaacaacacgcacagggcgaagctttagagccagatatggaagaagacgacgatcaggtcgac

SEQ ID NO: 9 - DNA, 72 bp, Artificial Sequence
  Human proinsulin and fusion partner
catatgaaaatcgaagaaggtaaactggcgggggacaatgacgacctcgacctggaagaagctttagtcgac

SEQ ID NO: 10 - DNA, 72 bp, Artificial Sequence
  Human proinsulin and fusion partner
catatgtctgaacaacacgcacagggcgcgggggacaatgacgacctcgacctggaagaagctttagtcgac

SEQ ID NO: 11 - DNA, 114 bp, Artificial Sequence
  Human proinsulin and fusion partner
catatgggcagcagccatcatcatcatcatcacagcagcgcgggggacaatgacgacctcgacctggaagaagctttagagccagatatggaagaagacgacgatcaggtcgac

SEQ ID NO: 12 - DNA, 87 bp, Artificial Sequence
  Human proinsulin and fusion partner
catatgaaaatcgaagaaggtaaactggcgggggacaatgtcctcctcgacctgatcttagctttagcgccaattatggaagtcgac

SEQ ID NO: 13 - DNA, 72 bp, Artificial Sequence
  Human proinsulin and fusion partner
catatgaaaatcgaagaaggtaaactggaagctttagtgccaattatggtagcagacgtcgctcaggtcgac

SEQ ID NO: 14 - DNA, 72 bp, Artificial Sequence
  Human proinsulin and fusion partner
catatgaaaatcgaagaaggtaaactggcgggggacaatgtcctcctcgacctgatcttagctttagtcgac

SEQ ID NO: 15 - DNA, 60 bp, Artificial Sequence
  Human proinsulin and fusion partner
catatgaaaatcgaagaaggtaaactggaagctttagagccagatatggaagaagtcgac

SEQ ID NO: 16 - DNA, 63 bp, Artificial Sequence
  Human proinsulin and fusion partner
catatgaaaatcgaagaaggtaaactggcgggggacaatgacgacctcgacctggaagtcgac

SEQ ID NO: 17 - DNA, 63 bp, Artificial Sequence
  Human proinsulin and fusion partner
catatgtctgaacaacacgcacagggcgcgggggacaatgacgacctcgacctggaagtcgac

SEQ ID NO: 18 - DNA, 279 bp, Artificial Sequence
  Human proinsulin and fusion partner
gtcgaccgtcgcttcgttaatcagcacctgtgcggctctcacctggtagaagctctgtacctggtttgcggtgaacgtggttttttctacaccccgaaaacccgtcgcgaggctgaagacctgcaggtaggtcaggttgaactgggcggtggtccgggtgcaggctctctgcagccgttggcgctggaaggttccctgcagaaacgtggcatcgttgaacaatgctgtactagcatctgctctctctaccagctggagaactattgtaactgaggatcc

SEQ ID NO: 19 - DNA, 549 bp, Artificial Sequence
  hGCSF DNA
gtcgacgacgacgacaaaacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctgaggatcc

SEQ ID NO: 20 - DNA, 600 bp, Artificial Sequence
  hGH DNA
gtcgacgacgacgacaaattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgcttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactacttaagaactacgggctgctctactgcttcaggaaggacatggacaaggtcgagacattcctgcgcatcgtgcagtgccgctctgtggagggcagctgtggcttctgaggatcc

SEQ ID NO: 21 - DNA, 369 bp, Artificial Sequence
  hBMP2 DNA
gtcgacgacgacgacaagcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctgaggatcc

SEQ ID NO: 22 - DNA, 105 bp, Artificial Sequence
  Sense primer
catatgggcagcagccatcatcatcatcatcacagcagcggcctggtgccgcgcggcagcgacatggcgggggacaatgacgacctcgacctggaagaagcttta

SEQ ID NO: 23 - DNA, 48 bp, Artificial Sequence
  Antisense primer
gtcgacctgatcgtcgtcttcttccatatctggctctaaagcttcttc

SEQ ID NO: 24 - DNA, 42 bp, Artificial Sequence
  Sense primer
catatggcgggggacaatgacgacctcgacctggaagaagct

SEQ ID NO: 25 - DNA, 48 bp, Artificial Sequence; identical to SEQ ID NO: 23
  Antisense primer

SEQ ID NO: 26 - DNA, 66 bp, Artificial Sequence
  Sense primer
catatgaaaatcgaagaaggtaaactggcgggggacaatgacgacctcgacctggaagaagcttta

SEQ ID NO: 27 - DNA, 48 bp, Artificial Sequence; identical to SEQ ID NO: 23
  Antisense primer

SEQ ID NO: 28 - DNA, 66 bp, Artificial Sequence
  Sense primer
catatgtctgaacaacacgcacagggcgcgggggacaatgacgacctcgacctggaagaagcttta

SEQ ID NO: 29 - DNA, 48 bp, Artificial Sequence; identical to SEQ ID NO: 23
  Antisense primer

SEQ ID NO: 30 - DNA, 66 bp, Artificial Sequence; identical to SEQ ID NO: 26
  Sense primer

SEQ ID NO: 31 - DNA, 33 bp, Artificial Sequence
  antisense primer
gtcgacttccatatctggctctaaagcttcttc

SEQ ID NO: 32 - DNA, 66 bp, Artificial Sequence; identical to SEQ ID NO: 28
  sense primer

SEQ ID NO: 33 - DNA, 33 bp, Artificial Sequence; identical to SEQ ID NO: 31
  antisense primer

SEQ ID NO: 34 - DNA, 45 bp, Artificial Sequence
  sense primer
catatgaaaatcgaagaaggtaaactggaagctttagagccagat

SEQ ID NO: 35 - DNA, 36 bp, Artificial Sequence
  antisense primer
gtcgacctgatcgtcgtcttcttccatatctggctc

SEQ ID NO: 36 - DNA, 45 bp, Artificial Sequence
  sense primer
catatgtctgaacaacacgcacagggcgaagctttagagccagat

SEQ ID NO: 37 - DNA, 36 bp, Artificial Sequence; identical to SEQ ID NO: 35
  antisense primer

SEQ ID NO: 38 - DNA, 57 bp, Artificial Sequence
  sense primer
catatgaaaatcgaagaaggtaaactggcgggggacaatgacgacctcgacctggaa

SEQ ID NO: 39 - DNA, 21 bp, Artificial Sequence
  antisense primer
gtcgactaaagcttcttccag

SEQ ID NO: 40 - DNA, 57 bp, Artificial Sequence
  sense primer
catatgtctgaacaacacgcacagggcgcgggggacaatgacgacctcgacctggaa

SEQ ID NO: 41 - DNA, 21 bp, Artificial Sequence
  antisense primer
gtcgactaaagcttcttccag

SEQ ID NO: 42 - DNA, 75 bp, Artificial Sequence
  sense primer
catatgggcagcagccatcatcatcatcatcacagcagcgcgggggacaatgacgacctcgacctggaagaagct

SEQ ID NO: 43 - DNA, 48 bp, Artificial Sequence; identical to SEQ ID NO: 23
  antisense primer

SEQ ID NO: 44 - DNA, 69 bp, Artificial Sequence
  sense primer
catatgaaaatcgaagaaggtaaactggcgggggacaatgtcctcctcgacctgatcttagctttagcg

SEQ ID NO: 45 - DNA, 30 bp, Artificial Sequence
  antisense primer
gtcgacttccataattggcgctaaagctaa

SEQ ID NO: 46 - DNA, 57 bp, Artificial Sequence
  sense primer
catatgaaaatcgaagaaggtaaactggaagctttagtgccaattatggtagcagac

SEQ ID NO: 47 - DNA, 30 bp, Artificial Sequence
  antisense primer
gtcgacctgagcgacgtctgctaccataat

SEQ ID NO: 48 - DNA, 57 bp, Artificial Sequence
  sense primer
catatgaaaatcgaagaaggtaaactggcgggggacaatgtcctcctcgacctgatc

SEQ ID NO: 49 - DNA, 21 bp, Artificial Sequence
  antisense primer
gtcgactaaagctaagatcag

SEQ ID NO: 50 - DNA, 45 bp, Artificial Sequence; identical to SEQ ID NO: 34
  sense primer

SEQ ID NO: 51 - DNA, 27 bp, Artificial Sequence
  antisense primer
gtcgacttcttccatatctggctctaa

SEQ ID NO: 52 - DNA, 48 bp, Artificial Sequence
  sense primer
catatgaaaatcgaagaaggtaaactggcgggggacaatgacgacctc

SEQ ID NO: 53 - DNA, 24 bp, Artificial Sequence
  antisense primer
gtcgacttccaggtcgaggtcgtc

SEQ ID NO: 54 - DNA, 48 bp, Artificial Sequence
  sense primer
catatgtctgaacaacacgcacagggcgcgggggacaatgacgacctc

SEQ ID NO: 55 - DNA, 24 bp, Artificial Sequence
  antisense primer
gtcgacttccaggtcgaggtcgtc

SEQ ID NO: 56 - DNA, 27 bp, Artificial Sequence
  sense primer
gtcgaccgtcgcttcgttaatcagcac

SEQ ID NO: 57 - DNA, 21 bp, Artificial Sequence
  antisense primer
ggatcctcagttacaatagtt

SEQ ID NO: 58 - DNA, 27 bp, Artificial Sequence
  sense primer
gtcgacgacgacgacaaaacccccctg

SEQ ID NO: 59 - DNA, 21 bp, Artificial Sequence
  antisense primer
ggatcctcagggctgggcaag

SEQ ID NO: 60 - DNA, 33 bp, Artificial Sequence
  sense primer
gtcgacgacgacgacaaattcccaaccattccc

SEQ ID NO: 61 - DNA, 24 bp, Artificial Sequence
  antisense primer
ggatcctcagaagccacagctgcc

SEQ ID NO: 62 - DNA, 33 bp, Artificial Sequence
  sense primer
gtcgacgacgacgacaagcaagccaaacacaaa

SEQ ID NO: 63 - DNA, 24 bp, Artificial Sequence
  antisense primer
ggatcctcagcgacacccacaacc

SEQ ID NO: 64 - protein, 44 aa, Artificial Sequence
  fusion partner A
MGSSHHHHHHSSGLVPRGSDMAGDNDDLDLEEALEPDMEEDDDQ

SEQ ID NO: 65 - protein, 24 aa, Artificial Sequence
  fusion partner A
MAGDNDDLDLEEALEPDMEEDDDQ

SEQ ID NO: 66 - protein, 31 aa, Artificial Sequence
  fusion partner A
MKIEEGKLAGDNDDLDLEEALEPDMEEDDDQ

SEQ ID NO: 67 - protein, 31 aa, Artificial Sequence
  fusion partner A
MSEQHAQGAGDNDDLDLEEALEPDMEEDDDQ

SEQ ID NO: 68 - protein, 26 aa, Artificial Sequence
  fusion partner A
MKIEEGKLAGDNDDLDLEEALEPDME

SEQ ID NO: 69 - protein, 26 aa, Artificial Sequence
  fusion partner A
MSEQHAQGAGDNDDLDLEEALEPDME

SEQ ID NO: 70 - protein, 21 aa, Artificial Sequence
  fusion partner A
MKIEEGKLEALEPDMEEDDDQ

SEQ ID NO: 71 - protein, 21 aa, Artificial Sequence
  fusion partner A
MSEQHAQGEALEPDMEEDDDQ

SEQ ID NO: 72 - protein, 21 aa, Artificial Sequence
  fusion partner A
MKIEEGKLAGDNDDLDLEEAL

SEQ ID NO: 73 - protein, 21 aa, Artificial Sequence
  fusion partner A
MSEQHAQGAGDNDDLDLEEAL

SEQ ID NO: 74 - protein, 35 aa, Artificial Sequence
  fusion partner A
MGSSHHHHHHSSAGDNDDLDLEEALEPDMEEDDDQ

SEQ ID NO: 75 - protein, 26 aa, Artificial Sequence
  fusion partner A
MKIEEGKLAGDNVLLDLILALAPIME

SEQ ID NO: 76 - protein, 21 aa, Artificial Sequence
  fusion partner A
MKIEEGKLEALVPIMVADVAQ

SEQ ID NO: 77 - protein, 21 aa, Artificial Sequence
  fusion partner A
MKIEEGKLAGDNVLLDLILAL

SEQ ID NO: 78 - protein, 17 aa, Artificial Sequence
  fusion partner A
MKIEEGKLEALEPDMEE

SEQ ID NO: 79 - protein, 18 aa, Artificial Sequence
  fusion partner A
MKIEEGKLAGDNDDLDLE

SEQ ID NO: 80 - protein, 18 aa, Artificial Sequence
  fusion partner A
MSEQHAQGAGDNDDLDLE

SEQ ID NO: 81 - protein, 86 aa, Artificial sequence
  Human proinsulin
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 82 - protein, 174 aa, Artificial sequence
  hGCSF
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 83 - protein, 191 aa, Artificial sequence [sequence omitted]

SEQ ID NO: 84 - protein, 114 aa, Artificial sequence
  hBMP2
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR